SEQUENCE LISTING (5 sequences)

SEQ ID NO: 1 - protein, 37 aa, Artificial Sequence
  synthetic
SVSEIQLMHNLGKHLNSMERVEWLRKKLQDVHNFVAL

SEQ ID NO: 2 - protein, 11 aa, Artificial Sequence
  synthetic
SVSEIQLMHNL

SEQ ID NO: 3 - protein, 26 aa, Artificial Sequence
  synthetic
GKHLNSMERVEWLRKKLQDVHNFVAL

SEQ ID NO: 4 - protein, 12 aa, Artificial Sequence
  synthetic
GKHLNSMERVEW

SEQ ID NO: 5 - protein, 14 aa, Artificial Sequence
  synthetic
LRKKLQDVHNFVAL

(19) United States
(12) Patent Application Publication
(10) Pub. No.: US 2011/0263509 A1
(43) Pub. Date: Oct. 27, 2011

(54) PREPARATION AND USE OF HIGH-PURITY HEMOPARATIDE (HPTH-1-37) FOR THE TREATMENT OF INFLAMMATORY SCALING DISEASES OF THE SKIN

(75) Inventors: Wolf-Georg Forssmann, Kleines Wiesental (DE); Dirk Teichmuller, Linsengericht-Geislitz (DE)

(73) Assignee: Haemopep Pharma GmbH, Hannover (DE)

(21) Appl. No.: 13/123,046

(22) PCT Filed: Oct. 7, 2009
(86) PCT No.: PCT/EP09/63017

§ 371 Date: Jul. 5, 2011

(30) Foreign Application Priority Data
Oct. 7, 2008 (EP) 08166024.3

(51) Int. Cl. A61K 38/16; A61P 17/06; C07K 1/00

(52) U.S. Cl. 514/18.7; 530/324

(57) ABSTRACT

Use of hPTH-1-37 having the amino acid sequence SVSEIQLMHNLGKHLNSMERVEWLRKKLQDVHNFVAL (SEQ ID No. 1) or one of its natural and pharmacologically compatible derivatives, especially amidated, acylated, phosphorylated and glycosylated derivatives, for preparing a medicament for the treatment of inflammatory scaling (erythematosquamous) diseases, especially psoriasis, wherein hPTH-1-37 (SEQ ID No. 1) is present in an amount of from 300 μg to 30 mg per gram of medicament.

The invention relates to the use of hPTH-1-37 for preparing a medicament for the treatment of inflammatory scaling diseases, the use of its derivatives for preparing a medicament for the treatment of inflammatory scaling diseases, a process for preparing hPTH-1-37, and a medicament comprising hPTH-1-37 or its derivatives.

Hemoparatide (hPTH-1-37) is the naturally occurring form of the processed parathyroid hormone (hPTH), which has been prepared from human blood (Hock et al., 1997). PTH is an essential peptide formed in the parathyroid gland that plays a crucial role in numerous metabolic functions. In particular, it regulates the cell proliferation and differentiation of keratinocytes in the skin.

When hPTH is lacking or deficient, severe diseases of the skin, such as inflammation, thickening and scaling, occur since PTH inhibits the cell proliferation of the immature keratinocytes and at the same time promotes the conversion thereof to mature keratinocytes that form the uppermost layer of the skin (Whitfield et al., 2004). Therefore, such inflammatory scaling (erythematosquamous) diseases can be treated by topical supply of PTH. The treatment of these diseases, which includes both the provision of a highly pure active substance and the application thereof to the skin, is described in the following and is provided by the invention.

As can be seen from the efficiency and concentration of hemoparatide in the blood plasma, this peptide is the most important bioactive form of PTH in the human body. Therefore, the above described effects of hPTH on the skin are not brought about by the complete molecule in the organism, but by its processed form hPTH-1-37.

U.S. Pat. No. 6,066,618 discloses a process for inhibiting the proliferation of mammal skin cells, wherein hPTH-1-34 is employed as an antiproliferative peptide. Further, DE-A-19508672 discloses cyclic parathyroid hormone fragments of, for example, hPTH(1-34), which may be employed for treating psoriasis, among others. Both disclosures relate to exogenous derivatives of human parathyroid hormone, and therefore, a tendency to undesirable effects and a poorer bioavailability is to be expected therein.

In addition, WO-A-2004/024758 discloses parathyroid hormone peptides from fish species. These are suitable, among others, for treating psoriasis.

US-A-2007/0117157 also describes the treatment of psoriasis with parathyroid hormone peptides from fish species. However, the PTH derivatives described therein have an amino acid homology of only 53% to human PTH and exhibit a clearly changed biological activity.

WO 02/28420 relates to processes for the regulation of cell differentiation and proliferation, for example, for the treatment of psoriasis by administering nucleic acid molecules coding for PTH.

WO-A-89/03873 relates to the regulation of cell proliferation by using peptides such as PTH(1-34). However, this application does not relate to PTH peptides 1-37.

WO 2005-A-007184 relates to cyclic analogues of hPTH.

WO-A-2008/150929 relates to a composition comprising hPTH 1-37 that can be administered topically, for treating psoriasis in particular galenic formulations.

Various manufacturers have performed the chemical synthesis of hemoparatide and thus supplied the product for preclinical and clinical examinations and studies. The synthesis of hPTH is not trivial, so that even products with a high degree of impurities involving incompletely synthesized peptides have been sold.

It is an object of the present invention to provide a highly pure form of hemoparatide for use as a therapeutic agent.

In addition to the requirements for the purity of hemoparatide, it is also required to find galenic forms that optimize the use of the highly pure peptide, are adapted to the respective status and goal of the treatment, and allow hemoparatide to be locally applied in inflammatory scaling (erythematosquamous) skin diseases. Such formulations for hemoparatide, also in a highly pure form, have not been available to date. Therefore, for current regulations, on the one hand, a sufficiently pure active ingredient must be available that, on the other hand, can be provided in appropriate galenic forms, can additionally be prepared on a commercial scale and thus can be considered as highly pure form meeting the highest demands. Thus, another object can be seen in providing the highly pure hPTH in an adequate galenic dosage form.

These objects are achieved by the use according to claim 1 and the process according to claim 3.

The present invention relates to the use of hPTH-1-37 having the amino acid sequence SVSEIQLMHNLGKHLNSMERVEWLRKKLQDVHNFVAL (SEQ ID No. 1) for preparing a medicament for the treatment of inflammatory scaling (erythematosquamous) diseases, especially psoriasis.

In one embodiment, the hPTH-1-37 has a molecular weight of 4401.13 Da.

The present invention further relates to derivatives of hPTH-1-37, namely its natural and pharmacologically compatible derivatives, especially amidated, acylated, phosphorylated and glycosylated derivatives, for preparing a medicament for the treatment of inflammatory scaling (erythematosquamous) diseases, especially psoriasis.

Another aspect of the invention is a process for preparing hPTH-1-37 or its derivatives, characterized in that these are prepared by a chemical synthesis from the partial sequences SVSEIQLMHNL (SEQ ID No. 2) and GKHLNSMERVEWLRKKLQDVHNFVAL (SEQ ID No. 3), or SVSEIQLMHNL(SEQ ID No. 2), GKHLNSMERVEW (SEQ ID No. 4) and LRKKLQDVHNFVAL (SEQ ID No. 5), and purified by chromatography.

In one embodiment of the invention, the hemoparatide (hPTH-1-37) or its derivatives are used for preparing a medicament in different galenic application forms, especially as a lyophilizate. In one embodiment of this medicament, a hydrophilic ointment, especially according to the German Pharmacopoeia, is used as a galenic application base. Surprisingly, it has been found that the active ingredient is very stable in this formulation, except for methionine-oxidized metabolites. These oxidized metabolites also occur as natural PTH forms in the blood plasma, but can be avoided by working under a nitrogen atmosphere, and are free from side effects, being naturally occurring endogenous derivatives.

Another aspect of the invention is a medicament containing from 300 micrograms to 30 milligrams of hPTH-1-37 per gram of formulation (SEQ ID No. 1), and/or from 300 micrograms to 30 milligrams of its derivatives.

In one embodiment, the formulation contains typical ointment bases into which the peptide hPTH-1-37 is incorporated. A typical ointment contains:

| | | | |
|---|---|---|---|
| Emulsifying cetyl stearyl alcohol | 15-25 | g, | especially 21 g |
| type A | | | |
| Low viscosity wax | 5-15 | g, | especially 10 g |
| Glycerol 85% | 3-8 | g, | especially 5 g |
| Potassium sorbate | 0.08-1.20 | g, | especially 0.14 g |
| Anhydrous citric acid | 0.01-1.5 | g, | especially 0.07 g |
| Benzalkonium chloride | 50-200 | mg, | especially 100 mg |
| EDTA | 50-200 | mg, | especially 100 mg |
| Purified water | ad 100 | mg | |

The amount of hPTH-1-37 can be from 0.001 to 1.0% by weight, based on the ointment base.

### DESCRIPTION OF THE FIGURES

FIG. 1 discloses an HPLC chromatogram of the highly pure hemoparatide.

FIG. 2 shows a MALDI (matrix assisted laser desorption/ionization)/MS spectrum of hPTH-1-37 final product.

FIG. 3 discloses a chromatogram of capillary zone electrophoresis of hPTH-1-37 final product.

FIGS. 4A and 4B disclose HPLC chromatograms of hPTH-1-37. FIG. 4A shows a chromatogram of freshly prepared hPTH-1-37. FIG. 4B is a chromatogram of hPTH-1-37 stored for nine months. Only very small amounts of methionine-oxidized metabolites can be detected (see satellite peaks). Further, FIG. 5 discloses a comparison between the HPLC chromatograms of freshly prepared hPTH-1-37 (A) and a commercially obtainable preparation. The surprisingly good quality can be seen from the LACK OF SATELLITE PEAKS IN % A as compared to 5B.

The preparation of the active ingredient and its formulation is described in the following:

Chemical Synthesis of hPTH-1-37

The peptides according to the invention are prepared by chemical synthesis in solution or on a solid support. For this purpose, different combinations of protecting groups, e.g., Fmoc- or Boc-protected amino acids, can be employed. In addition to the stepwise solid-phase peptide synthesis according to the classical Merrifield principle, the peptides according to the invention may also be prepared from the protected peptide fragment by a convergent synthesis.

hPTH-1-37 Having the Amino Acid Sequence

SVSEIQLMHNLGKHLNSMERVEWLRKKLQDVHNFVAL

was prepared by using Fmoc(fluorenylmethoxycarbonyl)-protected amino acids by a stepwise solid-phase synthesis. The synthesis was performed on a Wang resin loaded with Fmoc-leucine (0.69 mmol/g, 100-200 mesh) as a solid support. The activation of the Fmoc-amino acids, which were employed in a tenfold molar excess, was performed with [(2-(1H-benzotriazol-1-yl)-1,1,3,3-tetramethyluronium hexafluorophosphate] (HBTU) with addition of 1-hydroxybenzotriazole (HOBt) and diisopropyl ethyl amine (DIEA) in N-methyl-2-pyrrolidinone (NMP) at room temperature. Acylation reactions were typically performed for 45 minutes. The following amino acid derivatives were employed for synthesis: Fmoc-L-Ala, Fmoc-L-Arg(Pbf), Fmoc-L-Asn(Trt), Fmoc-L-Asp(OtBu), Fmoc-L-Glu(OtBu), Fmoc-L-Gln(Trt), Fmoc-Gly, Fmoc-L-His(Trt), Fmoc-L-Ile, Fmoc-L-Leu, Fmoc-L-Lys(Boc), Fmoc-L-Met, Fmoc-L-Phe, Fmoc-L-Ser(tBu), Fmoc-L-Trp(Boc) and Fmoc-L-Val. The temporary Fmoc protecting groups were cleaved off with 20% piperidine in N-methyl-2-pyrrolidinone within 2-10 minutes. After the removal of the Fmoc protecting group, the peptidyl resin was carefully and repeatedly washed with NMP and then dichloromethane, and dried. The dry resin was subsequently suspended in a freshly prepared mixture of trifluoroacetic acid, ethanedithiol and water (94:3:3 by volume, 20 ml per 1 g of peptidyl resin) to cleave off the peptide, and agitated for three hours. The mixture was filtered, the residue was washed with further cleaving mixture, and the combined filtrates were slowly added to a tenfold volume of cold tert-butyl ethyl ether with cooling. The precipitated deprotected peptidic material was stored at +4° C. over night and subsequently isolated by filtration or centrifugation and dried under vacuum.

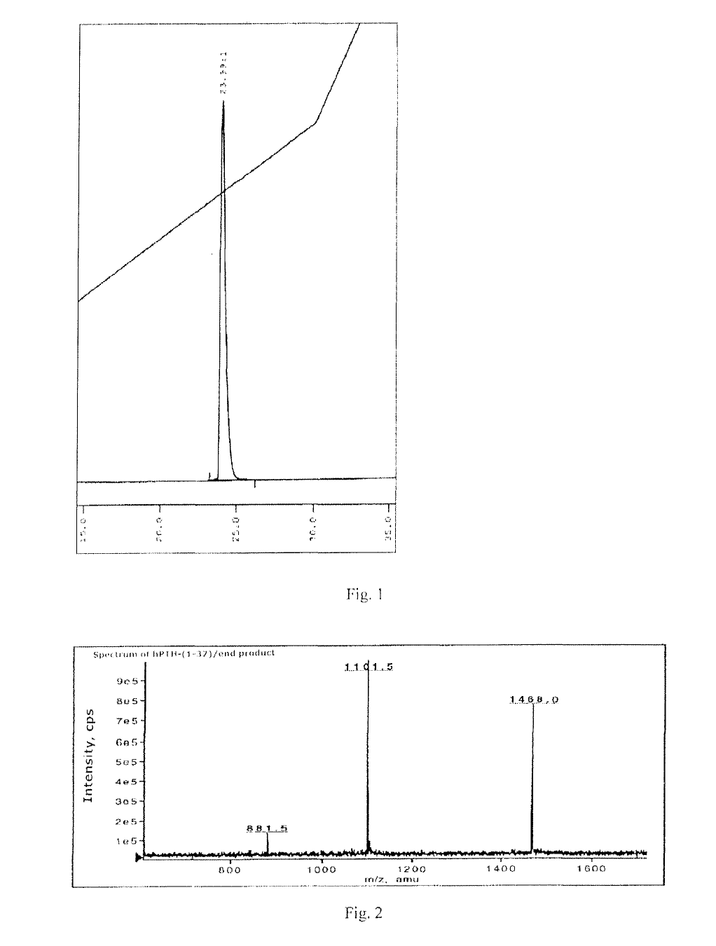

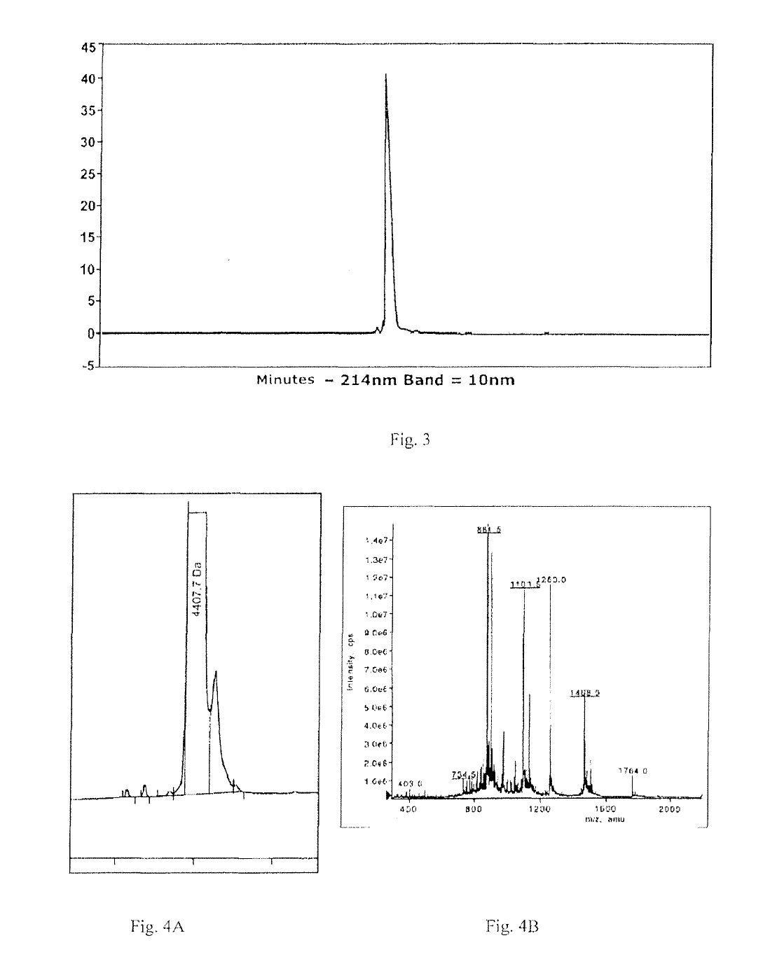

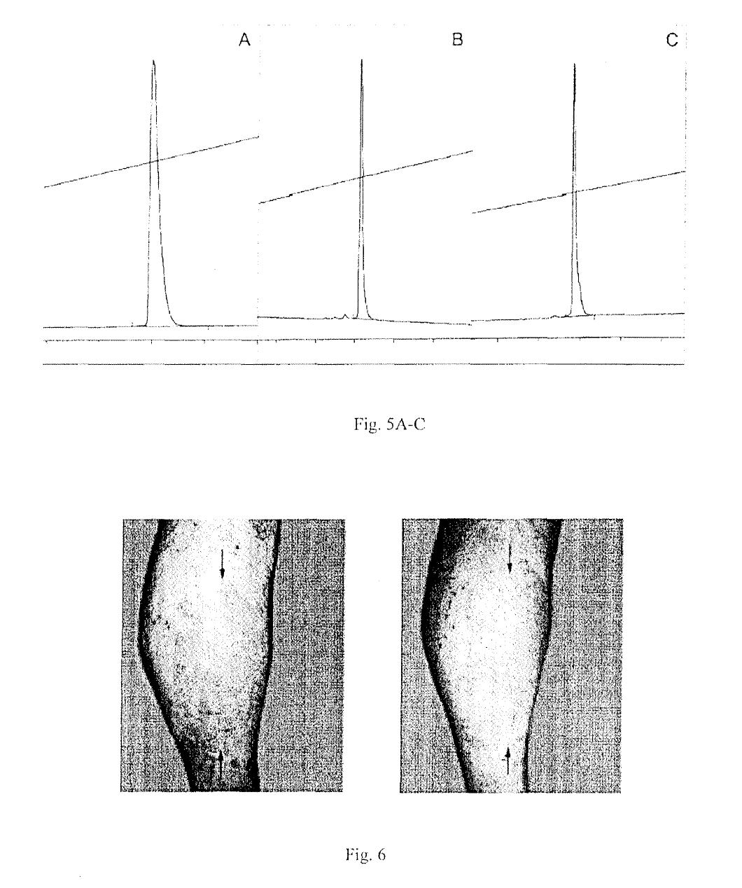

The raw peptide was dissolved in 10% acetic acid and purified by chromatography (Waters Prep-Pak C18, 47×300 mm; eluent A: 0.7% trifluoroacetic acid (TFA) in water; eluent B: 0.7% TFA in acetonitrile/water 4:1 (v/v); gradient: 35-55% eluent B in 40 minutes; detection: UV at 215 nm; flow rate: 40 ml/min). Fractions containing the product in sufficient purity (as determined by analytical HPLC) were combined and freeze-dried. The dry product was taken up in 10% acetic acid and subjected to chromatography in the presence of acetic acid/acetate to exchange the trifluoroacetate counter ion against acetate (Waters Prep-Pak C18, 47×300 mm, equilibrated with 0.1 M ammonium acetate solution; eluent A: 10% acetic acid in water; eluent B: 2% acetic acid in acetonitrile/water 4:1 (v/v); gradient: 10-60% eluent B in 40 minutes; detection: UV at 215 nm; flow rate: 40 ml/min). Fractions of sufficient purity were combined and freeze-dried. Dry end product was subsequently analyzed by RP-HPLC (FIG. 1), MALDI-MS (FIG. 2) and capillary zone electrophoresis (FIG. 3), and surprisingly a highly pure product was found whose specification is distinct from those of former syntheses by a clearly better quality (see FIGS. 4A and 4B). The surprisingly good quality can also be seen in a Table (Table 1) in comparison with a commercially available product (FIG. 5A vs. 5B).

Typical Characteristics of hPTH-1-37 and Highly Pure Hemoparatide Preparation

TABLE 1
The essential specifications used to evaluate the form of the peptide as
a highly pure product.

| | | | Specification of |
|---|---|---|---|
| | | Specification of | highly pure |
| | Method | hPTH-1-37 | hemoparatide |
| Molecular mass | Mass spectrometry | 4401.13 ± 1‰ | 4401.13 ± 0.8‰ |
| Peptide content | Amino acid analysis | ≧75% | not applicable |
| Acetate content | GC | ≦15% | not applicable |
| Purity | RP-HPLC (peak | ≧95% | ≧98% |
| | integration) | | |
| | Sum of by-products | ≦5% | ≦2% |
| | Individual by- | ≦0.5% | ≦0.4% |
| | products | | |
| Solubility | Dissolution time | ≦2 min | ≦2 min |
| Residual | By the method of | ≦15% | not applicable |
| moisture | Karl Fischer/GC | | |
| TFA | GC | ≦0.5% | not applicable |
| Acetonitrile | GC | ≦50 ppm | not applicable |

The high stability of the preparation of highly pure hemoparatide in lyophilized form at a temperature of +4° C. has been demonstrated by appropriate analytical methods, comparing fresh material (FIG. 4A) with material stored for nine months (FIG. 4B). After this storage period, only a small amount of methionine-oxidized metabolites appears.

### Preparation of a Novel Formulation of Hemoparatide

Creams on an oil-in-water basis in the aqueous phase of which the water-soluble hemoparatide has been incorporated are preferably suitable for galenic application.

Such a cream base is the hydrophilic ointment according to the German Pharmacopoeia (Unguentum emulsificans aquosum DAB).

TABLE 2
All the raw materials employed are in accordance with pharmaceutical
requirements.

| | | |
|---|---|---|
| Emulsifying cetyl stearyl alcohol type A | 21 | g |
| Low viscosity wax | 10 | g |
| Glycerol 85% | 5 | g |
| Potassium sorbate | 0.14 | g |
| Anhydrous citric acid | 0.07 | g |
| Benzalkonium chloride | 100 | mg |
| EDTA | 100 | mg |
| Purified water | ad 100 | g |

Hemoparatide was incorporated into this base in concentrations of 0.03% by weight, 0.1% by weight and 0.3% by weight.

Surprisingly, it has been found that the active ingredient is very stable in this formulation, except for methionine-oxidized metabolites. These oxidized metabolites also occur as natural PTH forms in the blood plasma, but can be avoided by working under a nitrogen atmosphere, and are free from side effects, being naturally occurring endogenous derivatives.

When the cream formulation is used, it is preferably applied thinly to the skin twice a day in galenic units as usual.

### Proof of Microbial Stability in Test for Efficacy of Preservation

In a test for efficacy of preservation, the microbial product stability of the formulation according to the invention was demonstrated according to pharmaceutical guidelines (pour plate method). The results show that all the requirements necessary for a skin medication are met.

### Tolerability and Safety of the Hemoparatide Cream Formulation

In humans, the high tolerability of the highly pure product for subcutaneous injection in an aqueous solution has been demonstrated by a phase I trial. Thus, formulations developed according to the invention for the topical application of the highly pure active ingredient hemoparatide can be considered highly safe and tolerable (see FIG. 6).

### Proof of Successful Treatment by Applying Hemoparatide Cream in Psoriasis:

The hPTH-1-37 cream was applied thinly twice a day on the lateral calf area on the right-hand side dorsally to the arrows:
1. The treated area has a surface area of about 4 times 10 cm.
2. In the morning and in the evening, about 250 mg each of ointment was uniformly applied by spreading the ointment with the forefinger.
3. The ointment contains a total of >20 μg of hPTH-1-37 per application, i.e., 40 μg per day.
4. The image on the left shows the right lower leg before the treatment, and the image on the right shows it after 14 days of treatment.

The thinning of the efflorescence and decline of inflammatory reaction after 14 days is clearly seen. The itching also subsides in the neighboring areas, and after a prolonged treatment and discontinuation of the medication, the effect is sustained for more than 4 weeks.

### REFERENCES

Hock D, Mägerlein M, Heine G, Ochlich P P, Forssmann W G (1997) Isolation and characterization of the bioactive circulating human parathyroid hormone, hPTH-1-37, FEBS Lett. 400: 221-225.
Whitfield J F (2004) Taming psoriatic keratinocytes—PTHs' uses go up another notch. JCB 93: 251-256.

1. Use of hPTH-1-37 having the amino acid sequence SVSEIQLMHNLGKHLNSMERVEWLRKKLQDVHNFVAL (SEQ ID No. 1) or one of its natural and pharmacologically compatible derivatives, especially amidated, acylated, phosphorylated and glycosylated derivatives, for preparing a medicament for the treatment of inflammatory scaling (erythematosquamous) diseases, especially psoriasis, wherein hPTH-1-37 (SEQ ID No. 1) is present in an amount of from 300 μg to 30 mg per gram of medicament.

2. The use according to claim 1, wherein hPTH-1-37 (SEQ ID No. 1) is present in the medicament in an amount of from 0.6 to 3% by weight.

3. A process for preparing hPTH-1-37 or its derivatives, characterized in that these are prepared by a chemical synthesis from the partial sequences SVSEIQLMHNL (SEQ ID No. 2) and GKHLNSMERVEWLRKKLQDVHNFVAL (SEQ ID No. 3), or SVSEIQLMHNL (SEQ ID No. 2), GKHLNSMERVEW (SEQ ID No. 4) and LRKKLQDVHNFVAL (SEQ ID No. 5), and purified by chromatography.

4. An ointment containing from 300 μg to 30 mg of hPTH-1-37 (SEQ ID No. 1) or of its natural and pharmacologically compatible derivatives, especially amidated, acylated, phosphorylated and glycosylated derivatives, per gram of ointment, especially from 6 mg to 30 mg of hPTH-1-37 per gram of ointment.

5. The ointment according to claim 4, wherein the ointment base is:

| | | | |
|---|---|---|---|
| Emulsifying cetyl stearyl alcohol | 15-25 | g, | especially 21 g |
| type A | | | |
| Low viscosity wax | 5-15 | g, | especially 10 g |
| Glycerol 85% | 3-8 | g, | especially 5 g |
| Potassium sorbate | 0.08-1.20 | g, | especially 0.14 g |
| Anhydrous citric acid | 0.01-1.5 | g, | especially 0.07 g |
| Benzalkonium chloride | 50-200 | mg, | especially 100 mg |
| EDTA | 50-200 | mg, | especially 100 mg |
| Purified water | ad 100 | mg | |
